(19) United States
(12) Patent Application Publication
(10) Pub. No.: US 2005/0137678 A1
(43) Pub. Date: Jun. 23, 2005

(54) Low profile resorbable stent

(75) Inventor: Ashish Varma, Galway (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc.

(21) Appl. No.: 10/740,806

(22) Filed: Dec. 22, 2003

(51) Int. Cl. A61F 2/06

(52) U.S. Cl. 623/1.15; 623/1.38; 264/108; 264/210.1; 264/290.2; 264/523; 264/531; 264/138; 264/400; 264/280; 264/564

(57) ABSTRACT

A low profile resorbable stent comprising an oriented, resorbable material, wherein said material has Young's Modulus and tensile strength in the oriented state greater than Young's modulus and tensile strength of unoriented material is disclosed. The low profile resorbable stent has a resorbable material with Young's modulus about 2-300 GPa and/or tensile strength 50-200 MPa. The resorbable material of the present invention is oriented such that the tensile strength and modulus are higher than the unoriented materials allowing for the low profile stent design. Also disclosed is a method of manufacturing a low profile resorbable stent. The method comprises providing an extrudate comprising a resorbable material, inducing molecular alignment in the extrudate to form an oriented extrudate and forming the stent from the oriented extrudate. The extrudate of resorbable material can be a sheet, tube or some other form. The sheet extrudate is stretched axially or biaxially to induce molecular alignment. The tubular extrudate is blow-molded to induce molecular alignment.

### BACKGROUND OF THE INVENTION

1. Field of the Invention

The present invention relates to the field of resorbable stents. Specifically, the present invention relates to resorbable stents having low profile and a process for their manufacture.

2. Related Art

Stents have gained acceptance in the medical community as a device capable of supporting body lumens, such as blood vessels, that have become weakened or are susceptible to closure. Typically, a stent is inserted into a vessel of a patient after an angioplasty procedure has been performed to partially open up the blocked/stenosed vessel thus allowing access for stent delivery and deployment. After the catheter used to perform angioplasty has been removed from the patient, a tubular stent, maintained in a small diameter delivery configuration at the distal end of a delivery catheter, is navigated through the vessels to the site of the stenosed area. Once positioned at the site of the stenosis, the stent is released from the delivery catheter and expanded radially to contact the inside surface of the vessel. The expanded stent provides a scaffold-like support structure to maintain the patency of the region of the vessel engaged by the stent, thereby promoting blood flow. Physicians may also elect to deploy a stent directly at the lesion rather than carrying out a pre-dilatation procedure. This approach requires stents that are highly deliverable i.e. have low profile and high flexibility.

Various types of endovascular stents have been proposed and used as a means for preventing restenosis. A typical stent is a tubular device capable of maintaining the lumen of the artery open. One example includes the metallic stents that have been designed and permanently implanted in arterial vessels. The metallic stents have low profile combined with high strength. Restenosis has been found to occur, however, in some cases despite the presence of the metallic stent. In addition, some implanted stents have been found to cause undesired local thrombosis. To address this, some patients receive anticoagulant and antiplatelet drugs to prevent local thrombosis or restenosis, however this prolongs the angioplasty treatment and increases its cost.

A number of non-metallic stents have been designed to address the concerns related to the use of permanently implanted metallic stents. U.S. Pat. No. 5,984,963 to Ryan, et al., discloses a polymeric stent made from resorbable polymers that degrades over time in the patient. U.S. Pat. No. 5,545,208 to Wolff, et al., discloses a polymeric prosthesis for insertion into a lumen to limit restenosis. The prosthesis carries restenosis-limiting drugs that are released as the prosthesis is resorbed. The use of resorbable polymers, however, has drawbacks that have limited the effectiveness of polymeric stents in solving the post-surgical problems associated with balloon angioplasty.

Polymeric stents are typically made from bioresorbable polymers. Materials and processes typically used to produce resorbable stents result in stents with low tensile strengths and low modulus, compared to metallic stents of similar dimensions. The limitations in mechanical strength of the resorbable stents can result in stent recoil after the stent has been inserted. This can lead to a reduction in luminal area and hence blood flow. In severe cases the vessel may completely re-occlude. In order to prevent the recoil, polymeric stents have been designed with thicker struts (which lead to higher profiles) or as composites to improve mechanical properties. The use of relatively thick struts makes polymeric stents stiffer and decreases their tendency to recoil, but a significant portion of the lumen of the artery can be occupied by the stent. This makes stent delivery more difficult and can cause a reduction in the area of flow through the lumen. A larger strut area also increases the level of injury to the vessel wall and this may lead to higher rates of restenosis i.e. re-occlusion of the vessel.

Considerable research has been undertaken to develop resorbable stents that are satisfactory alternatives to metallic stents and are usable as an adjunct to angioplasty. However, there remains a need for materials and processes to produce resorbable stents with high tensile strengths, high modulus and low profile.

### SUMMARY OF THE INVENTION

It has been found that low profile resorbable stents having enhanced properties can be produced by introducing molecular alignment or orientation in the resorbable materials used in stent production. The present invention, therefore, relates to a method of controlling the morphology of the oriented resorbable materials and a method of manufacturing a low profile stent comprising the oriented resorbable materials.

An embodiment of the present invention relates to a low profile resorbable stent comprising an oriented, resorbable material, wherein said material has Young's Modulus in the oriented state greater than Young's modulus of the same resorbable material in an unoriented state. Alternatively, said material has Young's Modulus and tensile strength in the oriented state greater than Young's modulus and tensile strength of the same resorbable material in an unoriented state. Resorbable stents of the present invention are produced comprising a resorbable material having Young's Modulus greater than about 2 GPa and preferably in the range of about 2-300 GPa. Alternatively, resorbable stents are produced comprising materials having a tensile strength greater than about 50 MPa and Young's modulus greater than about 2 GPa, or preferably having tensile strength about 50-200 MPa and Young's modulus about 2-300 GPa. The stents of the present invention optionally further comprise one or more of a biologically active agent, plasticizer and modifier.

In another embodiment, the present invention relates to a method of manufacturing a low profile resorbable stent. The method comprises providing an extrudate comprising a resorbable material, inducing molecular alignment in said extrudate to form an oriented extrudate and forming said stent from said oriented extrudate. The extrudate of resorbable material can be a sheet, tube or some other form. The sheet extrudate is stretched axially or biaxially to induce molecular alignment. The tubular extrudate is blow-molded to induce molecular alignment. The tubular extrudate may also be drawn over a tapered die.

It is to be understood that both the foregoing general description and the following detailed description are exemplary and explanatory and are intended to provide further explanation of the invention as claimed.

### DETAILED DESCRIPTION OF THE INVENTION

Reference will now be made in detail to the embodiments of the present invention, examples of which are illustrated in the accompanying drawings.

An embodiment of the present invention relates to a low profile resorbable stent comprising an oriented, resorbable material, wherein said material has Young's Modulus in the oriented state greater than Young's modulus of the same resorbable material in an unoriented state. Alternatively, said material has Young's Modulus and tensile strength in the oriented state greater than Young's modulus and tensile strength of the same resorbable material in an unoriented state. Resorbable is used herein to mean a material that dissolves over time. The process of dissolving can be by degradation, dissolution or by some other means by which the stent material dissolves into the body. Resorbable stents of the present invention are bioresorbable, or alternatively, biodegradable. Resorbable stents of the present invention comprise materials having a Young's modulus greater than about 2 GPa. Preferably, resorbable stents are produced that comprise materials having Young's modulus about 2-300 GPa.

As used herein, the term modulus, also known as the Young's modulus, is the stress per unit strain. The modulus is a measure of the stiffness of a material. Any method known to one of ordinary skill in the art can be used to measure modulus. For example, modulus can be measured using a tensile tester in accordance with methods well known in the art. Alternatively, a dynamic mechanical analyzer (DMA) is used to measure shear modulus, which can be converted to Young's modulus, as is well known to one skilled in the relevant art.

Tensile strength is the measure of the ability of a polymer to withstand pulling or expanding stresses. Resorbable stents of the present invention comprise materials having a tensile strength greater than about 50 MPa and Young's modulus greater than about 2 GPa. Preferably, resorbable stents are produced that comprise materials having tensile strength about 50-200 MPa and Young's modulus about 2-300 GPa. Tensile strength can be measured by any method known to one of ordinary skill in the art. One example is the testing method ASTM-D638-72 (available from ASTM International, West Conshohocken, Pa., 19428).

The resorbable stents of the present invention have a low profile. The low profile allows the practitioner to use the stent in a variety of body lumens. For example, stents of the present invention can be used in blood-carrying vessels such as arteries and veins. More specifically, vessels in which the stents can be used include cardiovascular, neurovascular and peripheral blood carrying vessels. By way of example, a resorbable stent of the present invention for use in a cardiovascular vessel has wall or strut thickness less than about 0.3 mm. Alternatively, the wall or strut thickness is about 0.05-0.25 mm, alternatively 0.08-0.15 mm. Stents for use in peripheral vessels can have the same or greater thickness. Stents for use in neurovascular vessels can have the same or lesser thickness.

Resorbable stents of the present invention comprise an oriented resorbable material. The term oriented is well known to one of ordinary skill in the art and is used herein to mean molecular alignment has been introduced into the material. Molecular orientation or alignment can be introduced in crystalline and amorphous phases of the material. Molecular orientation or alignment enhances the mechanical properties of the material. For example, introducing molecular alignment in a material increases the material's Young's modulus and tensile strength. One aspect of the present invention, therefore, is related to a method of inducing molecular alignment in a resorbable material to produce an oriented material, wherein the material has a greater Young's modulus and tensile strength than the unoriented material. The materials of the present invention can have any level of orientation or molecular alignment, so long as the material has higher modulus and tensile strength compared to the unoriented material. The enhanced mechanical properties of the oriented resorbable materials allow for the production of stents having high recoil resistance and low profile. Any method known to one skilled in the relevant art can be used to measure molecular alignment. For example, X-Ray analysis, can be used to determine the degree or amount of molecular alignment in the material. Alternatively, Fourier Transform Infrared (FTIR) spectroscopy is used, as is well known to one skilled in the relevant art.

Materials for use in the present invention include any resorbable material. In one example, the material comprises a resorbable polymer. Resorbable polymers for use in the present invention include but are not limited to polyesters, polyanhydrides, polyamides, polyurethanes, polyureas, polyethers, polysaccharides, polyamines, polyphosphates, polyphosphonates, polysulfonates, polysulfonamides, polyphosphazenes, a hydrogel, polylactides or polyglycolides. Specific examples of resorbable polymers include but are not limited to fibrin, collagen, polycaprolactone, poly(glycolic acid), poly(3-hydroxybutric acid), poly(d-lactic acid), poly(dl-lactic acid), poly(1-lactic acid) (PLLA), poly(lactide/glycolide) copolymers, poly(hydroxyvalerate), poly(hydroxy-varelate-co-hydroxybutyrate), or other PHAs, or other resorbable materials, e.g., protein cell matrices, plant and carbohydrate derivatives (sugars). Resorbable polymers of the present invention can be homopolymers, copolymers or a blend of two or more homopolymers or copolymers. Resorbable polymers of the present invention can have any molecular architecture and can be linear, branched, hyper-branched or dendritic, preferably they are linear or branched.

The resorbable polymers can be any molecular weight, as long as the material that comprises the resorbable polymer has Young's modulus about 2-300 GPa and/or tensile strength about 50-200 MPa. The molecular weight of the polymer effects the mechanical properties of the resulting stent. Resorbable polymers can range from a single repeat unit to about 10 million repeat units. More specifically, resorbable polymers can have molecular weights of about 10 Daltons to about 100,000,000 Daltons. Resorbable stents can comprise polymer compositions having a range or specific combination of ranges of molecular weights. Resorbable stents of the present invention comprise a single polymer, or alternatively, a blend of two or more different polymers. Specific preferred examples of resorbable polymers for use in the present invention include but are not limited to linear poly(1-lactic acid) and poly(glycolic acid) having molecular weights about 100,000-1,000,000 Daltons.

The resorbable stent optionally further comprises a plasticizer. Plasticizer is used herein to mean any material that can decrease the flexural modulus of a polymer. The plasticizer can influence the morphology of the polymer and can affect the melting temperature and glass transition temperature. Examples of plasticizers include, but are not limited to: small organic and inorganic molecules, oligomers and small molecular weight polymers (those having molecular weight less than about 50,000), highly-branched polymers and dendrimers. Specific examples include: ethylene glycol, diethylene glycol, triethylene glycol, oligomers of ethylene glycol, 2-ethylhexanol, isononyl alcohol, isodecyl alcohol, sorbitol, mannitol, oligomeric ethers such as oligomers of polyethylene glycol, including PEG-500, PEG 1000 and PEG-2000 and other biocompatible plasticizers.

The resorbable stent optionally further comprises a modifier. Modifier is used herein to refer to any material added to the polymer to affect the polymer's and stent's properties. Examples of modifiers for use in the invention include resorbable fillers, antioxidants, colorants, crosslinking agents and impact strength modifiers. The drugs and biologically active compounds and molecules.

The resorbable stent optionally further comprises a biologically active agent or drug. The agent or drug will be introduced into the body lumen as the stent is resorbed. Agents or drugs for use in the present invention include but are not limited to antiplatelet agents, calcium agonists, calcium antagonists, anticoagulant agents, antimitotic agents, antioxidants, antimetabolites, antithrombotic agents, anti-inflammatory agents, antiproliferative drugs, hypolipidemic drugs and angiogenic factors. Specific examples include but are not limited to glucocorticoids (e.g. dexamethasone, betamethasone), fibrin, heparin, hirudin, tocopherol, angiopeptin, aspirin, ACE inhibitors, growth factors and oligonucleotides.

Molecular orientation or alignment also effects the degradation rate of the material, and therefore, can effect the elution rate or release of a biological agent or drug. By introducing molecular alignment in the material, the elution rate of a drug will improve, allowing for the more controlled dosing of the patient.

Resorbable stents of the present invention can have any shape, geometry or construction. It is understood by one of ordinary skill in the art that the present invention is not limited to any one type of stent, but that the present invention can be applied to a variety of stent designs. By way of example, the present invention can be applied to the stent designs disclosed in U.S. Pat. No. 6,613,079; U.S. Pat. No. 6,331,189; U.S. Pat. No. 6,287,336; U.S. Pat. No. 6,156,062; U.S. Pat. No. 6,113,621; U.S. Pat. No. 5,984,963; U.S. Pat. No. 5,843,168, which are incorporated herein by reference.

In another embodiment, the present invention relates to a method of manufacturing a low profile resorbable stent. The method comprises providing an extrudate comprising a resorbable material, inducing molecular alignment in the extrudate to form an oriented extrudate and forming the stent from the oriented extrudate.

The process of extruding a material to form an extrudate is well known to one of ordinary skill in the art. Any method of extrusion, known to one of ordinary skill in the art, can be used to provide an extrudate. The extrudate can be any shape or size, specific examples include, but are not limited to sheets and tubes.

An extrudate in the form of a sheet can be produced by any extrusion method known to one of ordinary skill in the art. In one example, a resorbable material is first provided and mixed with other optional materials, for example a plasticizer, drug and modifier, to form a material composition. The composition is then extruded. It can be extruded through a flat die over a casting roll, through an annular die onto a sizing mandrel, between two or more rolls in a calendering process or by some other extrusion process. The temperature of the die and roll can be independently varied and controlled, preferably the temperature of the die or roll is not less than the glass transition temperature or melting temperature of the material composition. The extrusion temperature depends on the material being extruded. For example, poly(1-lactic acid) is extruded through a die or calendered between rolls at a temperature about 75-250° C. In another example, poly(glycolic acid) is extruded through a die or calendered between rolls at a temperature about 75-250° C. This process provides an extrudate in the form of a sheet. The particular extrusion method and parameters used during the extrusion process would be apparent to one skilled in the relevant art.

Molecular alignment is then introduced in the extruded sheet. Any alignment method known to one skilled in the relevant art can be used to introduce molecular alignment in the sheet. One particular example involves stretching the extruded sheet at a controlled temperature and controlled rate. The temperature and rate can be any temperature and rate that result in the introduction of molecular alignment in the extruded sheet. Preferably, the temperature is between the glass transition temperature and the melting temperature of the material. Any method can be used to stretch the sheet. For example, a machine is used, such as the Lab Stretcher Karo IV®, available from Brückner, in Schweinbach, Germany. The stretching process can be performed uniaxially or biaxially. Uniaxial stretching produces substantially uniaxial molecular orientation, whereas biaxial stretching produces biaxial molecular orientation. Biaxial stretching is performed sequentially, or alternatively, simultaneously. Bulk sheet properties such as sheet thickness are also controlled during the stretching process. Preferably, the sheet is stretched uniaxially to induce the maximum increase in tensile strength and modulus in the stretch direction. The draw ratio measures the relative degree of stretching between the stretched sheet and unstretched sheet. In the present invention, draw ratios can range from about 1.5 to about 10. The higher the draw ratio, the greater the amount of molecular alignment, and therefore, the greater the increase in tensile strength and modulus of the resorbable material. The amount of molecular alignment can be monitored before, during and after the stretching. Any method of monitoring the level of orientation can be used. For example, FTIR is used, as is well known to one skilled in the relevant art. This process provides an oriented extrudate in the form of a sheet.

An extrudate in the form of a tube can be produced by any extrusion method known to one skilled in the relevant art. Examples of extruders for use in the invention include single screw and double screw extruders that produce tube-shaped extrudates. The extrusion temperature depends on the material being extruded, and should be above the glass transition temperature of the material. For example, poly(1-lactic acid) is extruded at a temperature about 75-250° C. In another example, poly(glycolic acid) is extruded at a temperature about 75-250° C. The tubular extrudate can be cooled in a bath with a suitable fluid or in air. The tubular extrudate is a hollow cylindrical-shaped tube having a longitudinal axis.

Molecular alignment is then introduced in the extruded tube. Any method known to one of ordinary skill in the art can be used to introduce molecular alignment in the tube. One particular example involves introducing radial molecular alignment by blow-molding the tube at a temperature approximately between the glass transition temperature and the melting temperature. For example, a tubular extrudate comprising poly(1-lactic acid) is radially expanded or blow-molded at a temperature about 75-250° C. In another example, a tubular extrudate comprising poly(glycolic acid) is radially expanded or blow-molded at a temperature about 75-250° C. Any method of blow-molding the tubular extrudate can be used to induce the molecular alignment. In one example, a tubular extrudate is placed in a blow-molding machine and radially expanded. A suitable medium is used to expand the extrudate. Suitable medium can be a gas or liquid, or there can be no medium and the expansion is performed mechanically. The molecular alignment in the extrudate is related to the amount of expansion or draw ratio. The greater the amount of expansion, the greater the amount of molecular alignment and the greater the increase in tensile strength and modulus.

An alternative method of inducing molecular alignment in a tubular extrudate comprises drawing the tube over a tapered die. The drawing can be performed at any temperature, preferably at a temperature between the glass transition and melting temperature of the material. The degree of taper in the die controls the draw ratio, and hence, the level of molecular orientation in the tube. Increasing the degree of taper increases the level of orientation. Any taper degree can be used in the present invention, as long as the material after drawing has a Young's modulus greater than the undrawn material. In the present invention, tubular extrudates are expanded, using any method described herein, to a draw ratio between about 1.5-10.

The oriented extrudate is used to produce a low profile resorbable stent. Any method known to one of ordinary skill in the art can be used to produce the stent. For example, the oriented sheet may be used to design a stent comprising a ratcheting mechanism. Any type of ratcheting mechanism may be used. One example of a ratcheting mechanism for use in the present invention is disclosed in U.S. Pat. No. 5,984,963. In another example, the oriented sheet may be used to design a stent in the form of a spiral. One example of a spiral-formed stent for use in the present invention is disclosed in U.S. Pat. No. 6,156,062. A ratcheting mechanism can be introduced into the oriented sheet using a laser machining process, by using a pre-shaped die, or any other method known to one of ordinary skill in the art. A locking mechanism can also be introduced into the stent using the same methods listed above for introducing the ratcheting mechanism. The ratcheting and locking mechanisms help to further enhance the recoil resistance of the low profile resorbable stents. In a further example, a low profile resorbable stent is formed from an oriented tubular extrudate. The stent can be formed using any method known to one of ordinary skill in the art, for example, the tube is laser machined to a desired geometry.

The use of molecular alignment in the resorbable materials of the present invention serves to reduce the extrudate (e.g., the sheet or tubing) thickness required for a particular stent design. This in turn reduces the strut thickness. The reduction in strut thickness reduces the overall stent profile, all while maintaining high strength and recoil resistance.

It will be understood by those skilled in the art that various changes in form and details may be made therein without departing from the spirit and scope of the present invention as defined in the appended claims. Thus, the breadth and scope of the present invention should not be limited by any of the above-described exemplary embodiments, but should be defined only in accordance with the following claims and their equivalents.

1. A low profile resorbable stent comprising an oriented, resorbable material, wherein said material has Young's Modulus in the oriented state greater than Young's modulus of the same resorbable material in an unoriented state.

2. The stent of claim 1, wherein said material has Young's Modulus greater than about 2 GPa.

3. The stent of claim 1, wherein said material has Young's Modulus and tensile strength in the oriented state greater than Young's modulus and tensile strength of the same resorbable material in an unoriented state.

4. The stent of claim 3, wherein said material has Young's Modulus greater than about 2 GPa and a tensile strength greater than about 50 MPa.

5. The stent of claim 4, wherein said material has Young's Modulus about 2-300 GPa and tensile strength about 50-200 MPa.

6. The stent of claim 1, wherein said stent has cylindrical-shaped body.

7. The stent of claim 1, wherein said resorbable material is bioresorbable or biodegradable.

8. The stent of claim 1, wherein said material is a polyester, polyanhydride, polyamide, polyurethane, polyurea, polyether, polysaccharide, polyamine, polyphosphate, polyphosphonate, polysulfonate, polysulfonamide, polyphosphazene, hydrogel, polylactide, polyglycolide, protein cell matrix, or copolymer or polymer blend therof.

9. The stent of claim 8, wherein said material is fibrin, collagen, polycaprolactone, poly(glycolic acid), poly(1-lactic acid), poly(3-hydroxybutric acid), poly(dl-lactic acid), poly(d-lactic acid), poly(lactide/glycolide) copolymers, poly(hydroxyvalerate) or poly(hydroxyvalerate-co-hydroxybutyrate).

10. The stent of claim 1, further comprising a biologically active agent.

11. The stent of claim 10, wherein said agent is an antiplatelet agent, calcium agonist, calcium antagonist, anticoagulant agent, antimitotic agent, antioxidant, antimetabolite, antithrombotic agent, anti-inflammatory agent, antiproliferative drug, hypolipidemic drug, angiogenic factor, glucocorticoid, dexamethasone, betamethasone, fibrin, heparin, hirudin, tocopherol, angiopeptin, aspirin, ACE inhibitor, growth factors or oligonucleotide.

12. The stent of claim 1, further comprising a plasticizer.

13. The stent of claim 12, wherein said plasticizer is ethylene glycol, diethylene glycol, triethylene glycol, 2-ethylhexanol, isononyl alcohol, isodecyl alcohol, sorbitol, mannitol, PEG-500, PEG 1000 or PEG-2000.

14. The stent of claim 1, further comprising a resorbable modifier.

15. The stent of claim 14, wherein said resorbable modifier is a filler, antioxidant, colorant, crosslinking agent and impact strength modifier.

16-34. (canceled)

35. A low profile resorbable stent comprising a biaxially-oriented, resorbable material, wherein said material has Young's Modulus in the oriented state greater than Young's modulus of the same resorbable material in an unoriented state.
